(19) United States
(12) Patent Application Publication
(10) Pub. No.: US 2022/0338743 A1
(43) Pub. Date: Oct. 27, 2022

(54) ELECTRONIC DEVICE AND METHOD FOR PROVIDING HEALTH INFORMATION BASED ON ELECTROCARDIOGRAM IN ELECTRONIC DEVICE

(71) Applicant: Samsung Electronics Co., Ltd., Suwon-si (KR)

(72) Inventors: Hyejung SEO, Suwon-si (KR); Jeongmin PARK, Suwon-si (KR)

(21) Appl. No.: 17/712,761

(22) Filed: Apr. 4, 2022

Related U.S. Application Data
(63) Continuation of application No. PCT/KR2022/004150, filed on Mar. 24, 2022.

(30) Foreign Application Priority Data
Apr. 21, 2021 (KR) 10-2021-0051769

(51) Int. Cl. A61B 5/0205; G06K 9/62; G16H 40/63; A61B 5/361; A61B 5/339

(52) U.S. Cl. CPC A61B 5/0205; G06K 9/6288; G16H 40/63; A61B 5/361; A61B 5/339; A61B 5/021

(57) ABSTRACT

An electronic device is provided. The electronic device includes a first biometric sensor comprising a plurality of electrodes and a measurement sensor electrically connected with the plurality of electrodes, a display, a memory, and a processor configured to obtain first biometric sensing information including a first electrocardiogram waveform using the first biometric sensor, identify a suspected disease based on the first electrocardiogram waveform and a previously obtained second electrocardiogram waveform, obtain second biometric sensing information corresponding to the identified suspected disease, and display, on the display, suspected disease information obtained based on the second biometric sensing information.

### CROSS-REFERENCE TO RELATED APPLICATION(S)

This application is a continuation application, claiming priority under § 365(c), of an International application No. PCT/KR2022/004150, filed on Mar. 24, 2022, which is based on and claims the benefit of a Korean patent application number 10-2021-0051769, filed on Apr. 21, 2021, in the Korean Intellectual Property Office, the disclosure of which is incorporated by reference herein in its entirety.

### TECHNICAL FIELD

The disclosure relates to electronic devices and methods for providing health information in electronic devices.

### BACKGROUND ART

Recent electronic devices come in various form factors for user convenience purposes and in reduced size for easy carrying. Further, interest in health is increasing, and so is interest in exercise for keeping healthy.

Electronic devices have been developed to measure and utilize various biometric signals and provide various services for the user's health care or check on the user's health condition through measurement of various biometric signals. With the development of technology, sensors equipped in electronic devices to measure biometric signals are diversified, and so are functions using biometric signals.

The above information is presented as background information only to assist with an understanding of the disclosure. No determination has been made, and no assertion is made, as to whether any of the above might be applicable as prior art with regard to the disclosure.

### DISCLOSURE

### Technical Problem

The electronic device may sense an electrocardiogram among various biometric signals. The electrocardiogram may be an action current (or electrical signal) according to contraction and relaxation of the heart. The electronic device may sense and record electrical signals by the heart through electrodes attached to the body. For example, the electronic device may sense 12 lead electrocardiogram (ECGs) using 10 electrodes. As another example, the electronic device may sense fewer ECGs (e.g., lead 1) than 12 lead ECGs with fewer electrodes (e.g., 3 electrodes) than 10 electrodes due to its portable size limitations and costs.

The electronic device may provide health information through electrocardiogram analysis. For example, the health information may include a suspected disease (e.g., arrhythmia). In addition to arrhythmia, electrocardiograms may be associated with a variety of other suspected diseases, including hypoglycemia, dehydration, and electrolyte imbalance. However, it may be difficult to identify other various suspected diseases, such as hypoglycemia, dehydration, and electrolyte imbalance, than arrhythmia, using only electrocardiogram.

Aspects of the disclosure are to address at least the above-mentioned problems and/or disadvantages and to provide at least the advantages described below. Accordingly, an aspect of the disclosure is to provide an electronic device capable of providing information about various suspected diseases other than arrhythmia using electrocardiogram and additional biometric sensing information and a method for providing health information based on electrocardiogram.

Additional aspects will be set forth in part in the description which follows and, in part, will be apparent from the description, or may be learned by practice of the presented embodiments.

### Technical Solution

In accordance with an aspect of the disclosure, an electronic device is provided. The an electronic device includes a first biometric sensor comprising a plurality of electrodes and a measurement sensor electrically connected with the plurality of electrodes, a display, a memory, and a processor configured to obtain first biometric sensing information including a first electrocardiogram waveform using the first biometric sensor, identify a suspected disease based on the first electrocardiogram waveform and a previously obtained second electrocardiogram waveform, obtain second biometric sensing information corresponding to the identified suspected disease, and display, on the display, suspected disease information obtained based on the second biometric sensing information.

In accordance with another aspect of the disclosure, a method for providing electrocardiogram-based health information in an electronic device is provided. The method includes obtaining first biometric sensing information comprising a first electrocardiogram waveform using a first biometric sensor comprising a plurality of electrodes and a measurement sensor, identifying a suspected disease based on the first electrocardiogram waveform and a previously obtained second electrocardiogram waveform, obtaining second biometric sensing information corresponding to the identified suspected disease, and displaying, on a display, suspected disease information obtained based on the second biometric sensing information.

In accordance with another aspect of the disclosure, a non-volatile storage medium storing instructions is provided. The instructions are configured to, when executed by at least one processor, enable the at least one processor to perform at least one operation, the at least one operation comprising obtaining first biometric sensing information comprising a first electrocardiogram waveform using a first biometric sensor comprising a plurality of electrodes and a measurement sensor and a measurement module, identifying a suspected disease based on the first electrocardiogram waveform and a previously obtained second electrocardiogram waveform, obtaining second biometric sensing information corresponding to the identified suspected disease, and displaying, on a display, suspected disease information obtained based on the second biometric sensing information.

### Advantageous Effects

According to various embodiments, the electronic device may identify a suspected disease by obtaining additional biometric sensing information (e.g., second biometric sensing information) corresponding to the suspected disease obtained from a first biometric sensing module (e.g., electrocardiogram sensing information) to identify the suspected disease, thereby providing information regarding various suspected diseases based on electrocardiogram.

Other aspects, advantages, and salient features of the disclosure will become apparent to those skilled in the art from the following detailed description, which, taken in conjunction with the annexed drawings, discloses various embodiments of the disclosure.

### DESCRIPTION OF THE DRAWINGS

The above and other aspects, features, and advantages of certain embodiments of the disclosure will be more apparent from the following description taken in conjunction with the accompanying drawings, in which:

FIG. 1 is a view illustrating a network environment according to an embodiment of the disclosure;

FIG. 2 is a block diagram illustrating an electronic device according to an embodiment of the disclosure;

FIG. 3A and FIG. 3B are views illustrating an example in which an electronic device is implemented as a wearable device according to an embodiment of the disclosure;

FIG. 4 is a view illustrating an electrocardiogram waveform based on heartbeat cycle according to an embodiment of the disclosure;

FIG. 5 is a view illustrating a change in electrocardiogram waveform due to an increase in blood potassium concentration according to an embodiment of the disclosure;

FIG. 6 is a flowchart illustrating an operation of providing electrocardiogram-based health information in an electronic device according to an embodiment of the disclosure;

FIG. 7 is a flowchart illustrating an operation of providing health information based on arrhythmia in an electronic device according to an embodiment of the disclosure;

FIG. 8 is a view illustrating an example of providing suspected disease information using biometric sensing information obtained from a sensor module included in an electronic device, an external electronic device, and an external server, in the electronic device according to an embodiment of the disclosure; and

FIG. 9 is a view illustrating an example of an operation of identifying suspected disease information by a plurality of parameter sets based on electrocardiogram in an electronic device according to an embodiment of the disclosure.

Throughout the drawings, it should be noted that like reference numbers are used to depict the same or similar elements, features, and structures.

### MODE FOR INVENTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings, but, are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural references unless the context clearly dictates otherwise. Thus, for example, reference to “a component surface” includes reference to one or more of such surfaces.

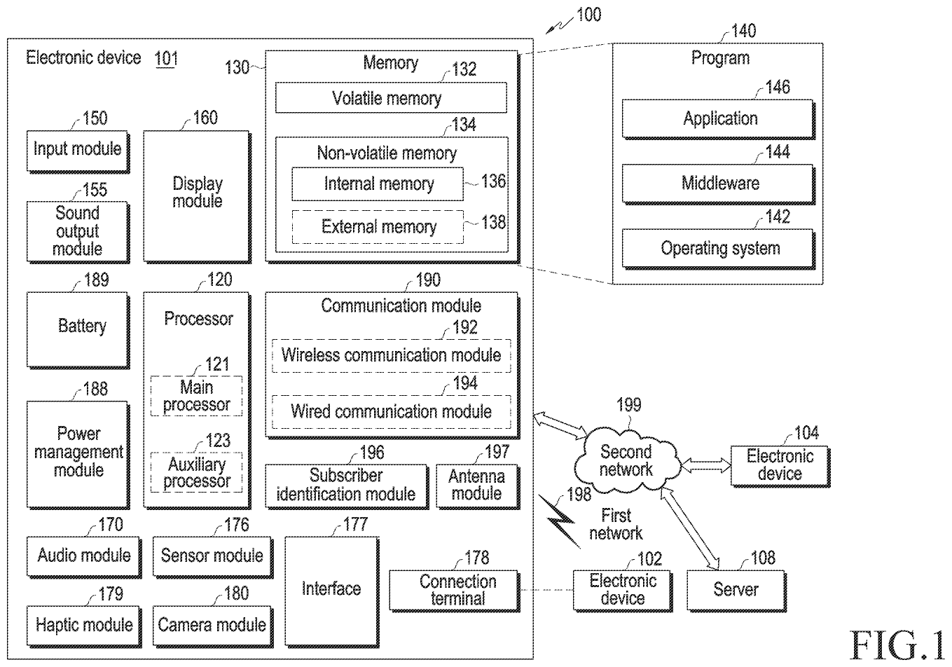

FIG. 1 is a block diagram illustrating an electronic device 101 in a network environment 100 according to an embodiment of the disclosure.

Referring to FIG. 1, the electronic device 101 in the network environment 100 may communicate with at least one of an electronic device 102 via a first network 198 (e.g., a short-range wireless communication network), or an electronic device 104 or a server 108 via a second network 199 (e.g., a long-range wireless communication network). According to an embodiment, the electronic device 101 may communicate with the electronic device 104 via the server 108. According to an embodiment, the electronic device 101 may include a processor 120, memory 130, an input module 150, a sound output module 155, a display module 160, an audio module 170, a sensor module 176, an interface 177, a connecting terminal 178, a haptic module 179, a camera module 180, a power management module 188, a battery 189, a communication module 190, a subscriber identification module (SIM) 196, or an antenna module 197. In some embodiments, at least one (e.g., the connecting terminal 178) of the components may be omitted from the electronic device 101, or one or more other components may be added in the electronic device 101. According to an embodiment, some (e.g., the sensor module 176, the camera module 180, or the antenna module 197) of the components may be integrated into a single component (e.g., the display module 160).

The processor 120 may execute, for example, software (e.g., a program 140) to control at least one other component (e.g., a hardware or software component) of the electronic device 101 coupled with the processor 120, and may perform various data processing or computation. According to one embodiment, as at least part of the data processing or computation, the processor 120 may store a command or data received from another component (e.g., the sensor module 176 or the communication module 190) in volatile memory 132, process the command or the data stored in the volatile memory 132, and store resulting data in non-volatile memory 134. According to an embodiment, the processor 120 may include a main processor 121 (e.g., a central processing unit (CPU) or an application processor (AP)), or an auxiliary processor 123 (e.g., a graphics processing unit (GPU), a neural processing unit (NPU), an image signal processor (ISP), a sensor hub processor, or a communication processor (CP)) that is operable independently from, or in conjunction with, the main processor 121. For example, when the electronic device 101 includes the main processor 121 and the auxiliary processor 123, the auxiliary processor 123 may be configured to use lower power than the main processor 121 or to be specified for a designated function. The auxiliary processor 123 may be implemented as separate from, or as part of the main processor 121.

The auxiliary processor 123 may control at least some of functions or states related to at least one component (e.g., the display module 160, the sensor module 176, or the communication module 190) among the components of the electronic device 101, instead of the main processor 121 while the main processor 121 is in an inactive (e.g., sleep) state, or together with the main processor 121 while the main processor 121 is in an active state (e.g., executing an application). According to an embodiment, the auxiliary processor 123 (e.g., an image signal processor or a communication processor) may be implemented as part of another component (e.g., the camera module 180 or the communication module 190) functionally related to the auxiliary processor 123. According to an embodiment, the auxiliary processor 123 (e.g., the neural processing unit) may include a hardware structure specified for artificial intelligence model processing. The artificial intelligence model may be generated via machine learning. Such learning may be performed, e.g., by the electronic device 101 where the artificial intelligence is performed or via a separate server (e.g., the server 108). Learning algorithms may include, but are not limited to, e.g., supervised learning, unsupervised learning, semi-supervised learning, or reinforcement learning. The artificial intelligence model may include a plurality of artificial neural network layers. The artificial neural network may be a deep neural network (DNN), a convolutional neural network (CNN), a recurrent neural network (RNN), a restricted Boltzmann machine (RBM), a deep belief network (DBN), a bidirectional recurrent deep neural network (BRDNN), deep Q-network or a combination of two or more thereof but is not limited thereto. The artificial intelligence model may, additionally or alternatively, include a software structure other than the hardware structure.

The memory 130 may store various data used by at least one component (e.g., the processor 120 or the sensor module 176) of the electronic device 101. The various data may include, for example, software (e.g., the program 140) and input data or output data for a command related thereto. The memory 130 may include the volatile memory 132 or the non-volatile memory 134.

The program 140 may be stored in the memory 130 as software, and may include, for example, an operating system (OS) 142, middleware 144, or an application 146.

The input module 150 may receive a command or data to be used by another component (e.g., the processor 120) of the electronic device 101, from the outside (e.g., a user) of the electronic device 101. The input module 150 may include, for example, a microphone, a mouse, a keyboard, keys (e.g., buttons), or a digital pen (e.g., a stylus pen).

The sound output module 155 may output sound signals to the outside of the electronic device 101. The sound output module 155 may include, for example, a speaker or a receiver. The speaker may be used for general purposes, such as playing multimedia or playing record. The receiver may be used for receiving incoming calls. According to an embodiment, the receiver may be implemented as separate from, or as part of the speaker.

The display module 160 may visually provide information to the outside (e.g., a user) of the electronic device 101. The display 160 may include, for example, a display, a hologram device, or a projector and control circuitry to control a corresponding one of the display, hologram device, and projector. According to an embodiment, the display 160 may include a touch sensor configured to detect a touch, or a pressure sensor configured to measure the intensity of a force generated by the touch.

The audio module 170 may convert a sound into an electrical signal and vice versa. According to an embodiment, the audio module 170 may obtain the sound via the input module 150, or output the sound via the sound output module 155 or a headphone of an external electronic device (e.g., an electronic device 102) directly (e.g., wiredly) or wirelessly coupled with the electronic device 101.

The sensor module 176 may detect an operational state (e.g., power or temperature) of the electronic device 101 or an environmental state (e.g., a state of a user) external to the electronic device 101, and then generate an electrical signal or data value corresponding to the detected state. According to an embodiment, the sensor module 176 may include, for example, a gesture sensor, a gyro sensor, an atmospheric pressure sensor, a magnetic sensor, an acceleration sensor, a grip sensor, a proximity sensor, a color sensor, an infrared (IR) sensor, a biometric sensor, a temperature sensor, a humidity sensor, or an illuminance sensor.

The interface 177 may support one or more specified protocols to be used for the electronic device 101 to be coupled with the external electronic device (e.g., the electronic device 102) directly (e.g., wiredly) or wirelessly. According to an embodiment, the interface 177 may include, for example, a high definition multimedia interface (HDMI), a universal serial bus (USB) interface, a secure digital (SD) card interface, or an audio interface.

A connecting terminal 178 may include a connector via which the electronic device 101 may be physically connected with the external electronic device (e.g., the electronic device 102). According to an embodiment, the connecting terminal 178 may include, for example, a HDMI connector, a USB connector, a SD card connector, or an audio connector (e.g., a headphone connector).

The haptic module 179 may convert an electrical signal into a mechanical stimulus (e.g., a vibration or motion) or electrical stimulus which may be recognized by a user via his tactile sensation or kinesthetic sensation. According to an embodiment, the haptic module 179 may include, for example, a motor, a piezoelectric element, or an electric stimulator.

The camera module 180 may capture a still image or moving images. According to an embodiment, the camera module 180 may include one or more lenses, image sensors, image signal processors, or flashes.

The power management module 188 may manage power supplied to the electronic device 101. According to one embodiment, the power management module 188 may be implemented as at least part of, for example, a power management integrated circuit (PMIC).

The battery 189 may supply power to at least one component of the electronic device 101. According to an embodiment, the battery 189 may include, for example, a primary cell which is not rechargeable, a secondary cell which is rechargeable, or a fuel cell.

The communication module 190 may support establishing a direct (e.g., wired) communication channel or a wireless communication channel between the electronic device 101 and the external electronic device (e.g., the electronic device 102, the electronic device 104, or the server 108) and performing communication via the established communication channel. The communication module 190 may include one or more communication processors that are operable independently from the processor 120 (e.g., the application processor (AP)) and supports a direct (e.g., wired) communication or a wireless communication. According to an embodiment, the communication module 190 may include a wireless communication module 192 (e.g., a cellular communication module, a short-range wireless communication module, or a global navigation satellite system (GNSS) communication module) or a wired communication module 194 (e.g., a local area network (LAN) communication module or a power line communication (PLC) module). A corresponding one of these communication modules may communicate with the external electronic device 104 via a first network 198 (e.g., a short-range communication network, such as Bluetooth™, wireless-fidelity (Wi-Fi) direct, or infrared data association (IrDA)) or a second network 199 (e.g., a long-range communication network, such as a legacy cellular network, a 5th generation (5G) network, a next-generation communication network, the Internet, or a computer network (e.g., local area network (LAN) or wide area network (WAN)). These various types of communication modules may be implemented as a single component (e.g., a single chip), or may be implemented as multi components (e.g., multi chips) separate from each other. The wireless communication module 192 may identify or authenticate the electronic device 101 in a communication network, such as the first network 198 or the second network 199, using subscriber information (e.g., international mobile subscriber identity (IMSI)) stored in the subscriber identification module 196.

The wireless communication module 192 may support a 5G network, after a 4th generation (4G) network, and next-generation communication technology, e.g., new radio (NR) access technology. The NR access technology may support enhanced mobile broadband (eMBB), massive machine type communications (mMTC), or ultra-reliable and low-latency communications (URLLC). The wireless communication module 192 may support a high-frequency band (e.g., the mmWave band) to achieve, e.g., a high data transmission rate. The wireless communication module 192 may support various technologies for securing performance on a high-frequency band, such as, e.g., beamforming, massive multiple-input and multiple-output (massive MIMO), full dimensional MIMO (FD-MIMO), array antenna, analog beam-forming, or large scale antenna. The wireless communication module 192 may support various requirements specified in the electronic device 101, an external electronic device (e.g., the electronic device 104), or a network system (e.g., the second network 199). According to an embodiment, the wireless communication module 192 may support a peak data rate (e.g., 20 Gbps or more) for implementing eMBB, loss coverage (e.g., 164 dB or less) for implementing mMTC, or U-plane latency (e.g., 0.5 ms or less for each of downlink (DL) and uplink (UL), or a round trip of 1 ms or less) for implementing URLLC.

The antenna module 197 may transmit or receive a signal or power to or from the outside (e.g., the external electronic device). According to an embodiment, the antenna module 197 may include one antenna including a radiator formed of a conductor or conductive pattern formed on a substrate (e.g., a printed circuit board (PCB)). According to an embodiment, the antenna module 197 may include a plurality of antennas (e.g., an antenna array). In this case, at least one antenna appropriate for a communication scheme used in a communication network, such as the first network 198 or the second network 199, may be selected from the plurality of antennas by, e.g., the communication module 190. The signal or the power may then be transmitted or received between the communication module 190 and the external electronic device via the selected at least one antenna. According to an embodiment, other parts (e.g., radio frequency integrated circuit (RFIC)) than the radiator may be further formed as part of the antenna module 197.

According to various embodiments, the antenna module 197 may form a mmWave antenna module. According to an embodiment, the mmWave antenna module may include a printed circuit board, a RFIC disposed on a first surface (e.g., the bottom surface) of the printed circuit board, or adjacent to the first surface and capable of supporting a designated high-frequency band (e.g., the mmWave band), and a plurality of antennas (e.g., array antennas) disposed on a second surface (e.g., the top or a side surface) of the printed circuit board, or adjacent to the second surface and capable of transmitting or receiving signals of the designated high-frequency band.

At least some of the above-described components may be coupled mutually and communicate signals (e.g., commands or data) therebetween via an inter-peripheral communication scheme (e.g., a bus, general purpose input and output (GPIO), serial peripheral interface (SPI), or mobile industry processor interface (MIPI)).

According to an embodiment, commands or data may be transmitted or received between the electronic device 101 and the external electronic device 104 via the server 108 coupled with the second network 199. The external electronic devices 102 or 104 each may be a device of the same or a different type from the electronic device 101. According to an embodiment, all or some of operations to be executed at the electronic device 101 may be executed at one or more of the external electronic devices 102, 104, or 108. For example, if the electronic device 101 should perform a function or a service automatically, or in response to a request from a user or another device, the electronic device 101, instead of, or in addition to, executing the function or the service, may request the one or more external electronic devices to perform at least part of the function or the service. The one or more external electronic devices receiving the request may perform the at least part of the function or the service requested, or an additional function or an additional service related to the request, and transfer an outcome of the performing to the electronic device 101. The electronic device 101 may provide the outcome, with or without further processing of the outcome, as at least part of a reply to the request. To that end, a cloud computing, distributed computing, mobile edge computing (MEC), or client-server computing technology may be used, for example. The electronic device 101 may provide ultra low-latency services using, e.g., distributed computing or mobile edge computing. In another embodiment, the external electronic device 104 may include an internet-of-things (IoT) device. The server 108 may be an intelligent server using machine learning and/or a neural network. According to an embodiment, the external electronic device 104 or the server 108 may be included in the second network 199. The electronic device 101 may be applied to intelligent services (e.g., smart home, smart city, smart car, or health-care) based on 5G communication technology or IoT-related technology.

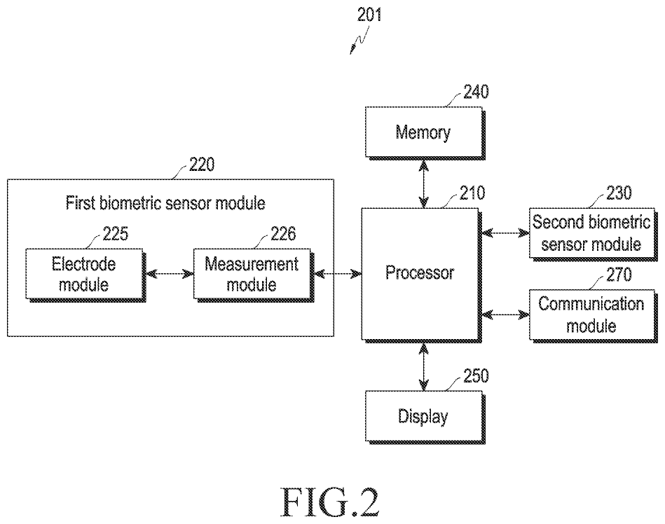

FIG. 2 is a block diagram illustrating an electronic device according to an embodiment of the disclosure.

Referring to FIG. 2, according to an embodiment, an electronic device 201 (e.g., the electronic device 101 of FIG. 1) may include at least one processor (hereinafter, a processor) 210, a first biometric sensor module 220, a second biometric sensor module 230, a memory 240, a display 250, and/or a communication module 270. The electronic device 201 is not limited thereto and may add more components or exclude some of the above-described components. According to an embodiment, the electronic device 201 may include the whole or part of the electronic device 101 of FIG. 1.

The first sensor module 220 according to an embodiment may include an electrode module 225 (or a sensor interface or sensor die) and a measurement module 226 (a processing unit or an integrated chip (IC)).

The electrode module 225 according to an embodiment may include a plurality of electrodes (e.g., at least two or more electrodes). For example, each of the at least two or more electrodes may be connected with a designated connection point of the human body.

The measurement module 226 according to an embodiment may include at least one switch and a current circuit and may be electrically connected with the electrode module 225 and the processor 210. According to an embodiment, at least one switch of the measurement module 226 may be connected with at least two or more electrodes to operate so that each of the at least two or more electrodes connects to or disconnects from a current circuit. When each of the at least two or more electrodes is connected with a current circuit, an input current may be applied, and an electrical signal may be measured based on the resistance and/or voltage formed between at least two or more electrodes based on the input current. For example, the first biometric sensor module 220 may include an ECG sensor.

The second biometric sensor module 230 according to an embodiment may include at least one other biometric sensor module (e.g., at least one second biometric sensor) related to biometric signal detection in addition to the ECG sensor. For example, the second biometric sensor module 230 may use the electrode module 225 of the first biometric sensor module 220 or a separate electrode module (not shown) and may include a sensing processor (e.g., a measurement module (processing unit or integrated chip (IC)) and may perform biometric signal sensing and processing. For example, the at least one other biometric sensor may include a photoplethysmography (PPG) sensor, a galvanic skin response sensor (GSR) sensor, an electrodermal activity (EDA) sensor capable of GSR sensing, a ballistocardiogram (BCG) sensor, a photo sensor, a sweat sensor for sensing hydration or dehydration, an iris sensor, and/or a body temperature sensor. According to an embodiment, the at least one other biometric sensor may further include a biometric sensor associated with identification of a suspected disease associated with electrocardiogram in addition to the aforementioned biometric sensors. For example, in addition to the ECG sensor, the at least one other biometric sensor may further include a sensor capable of sensing a pulse, sweat, blood, urine, and/or the state of or a change in the iris. At least one other biometric sensor according to an embodiment may be connected with at least some of the plurality of (at least two or more) electrodes of the electrode module 222 or have a separate electrode module to sense a biometric signal. For example, the ECG sensor may be connected with at least some of the at least two or more electrodes to sense an electrocardiogram, and at least one other biometric sensor may be connected with at least some of the at least two or more electrodes or may be connected using a separate electrode module or without connection to an electrode, to sense pulse, sweat, blood, urine, and/or state of or change in the state of the iris.

The processor 210 according to an embodiment may be electrically connected with the first biometric sensor module 220, the second sensor biometric sensor module 230, the memory 240, the display 250, and/or the communication module 270.

The processor 210 according to an embodiment may obtain a first electrocardiogram waveform using the first biometric sensor module 220 (e.g., an ECG sensor) and may identify the presence or absence of an arrhythmia (e.g., atrial fibrillation) based on the obtained first electrocardiogram waveform. For example, atrial fibrillation may be a condition in which the contraction of the atrium is lost during arrhythmia and occurs irregularly. For example, an arrhythmia may be accompanied with conditions other than atrial fibrillation.

When an arrhythmia is not identified, the processor 210 according to an embodiment may identify whether electrocardiogram-associated symptoms are present. For example, electrocardiogram-associated symptoms may include rapid heartbeat, skipping heartbeats, fatigue, shortness of breath, chest pain, tightness, fainting, and/or dizziness. For example, at least some of the electrocardiogram-associated symptoms may be associated with suspected diseases. For example, dizziness, increased heart rate, and fainting may be associated with hypoglycemia and dehydration among suspected diseases.

The processor 210 according to an embodiment may provide information indicating that the electrocardiogram is normal when an arrhythmia is not identified and there are no electrocardiogram-associated symptoms. When the electrocardiogram is normal, the processor 210 according to an embodiment may store the ECG waveform in the normal state in the memory 240. For example, the processor 210 may accrue and store the ECG waveforms in the normal state. According to an embodiment, the processor may learn or additionally process (e.g., superimpose) the accrued and stored ECG waveforms to obtain and store a representative normal ECG waveform representing the normal state.

When a presence of an arrhythmia and a presence of an ECG-associated symptom are identified or absence of the arrhythmia and the presence of the ECG-associated symptom are identified, the processor 210 according to an embodiment may identify the suspected disease by analyzing ECG factors (e.g., parameters) of ECG waveform based on the measured first electrocardiogram waveform and the previously obtained second electrocardiogram waveform (normal electrocardiogram waveform or representative electrocardiogram waveform). For example, the parameters may include a plurality of feature points (e.g., P-wave, QRS complex, T-wave) or sections associated with a plurality of feature points (e.g., segments or duration associated with P-wave, QRS complex, and/or T-wave). For example, the processor 210 may analyze all of the parameters or a parameter set obtained by combining at least some parameters from all of the parameters. For example, the parameter set may be designated as a parameter set associated with a suspected disease for each suspected disease that may be suspected in the ECG waveform. For example, a first parameter set associated with a first suspected disease, a second parameter set associated with a second suspected disease, a third parameter set associated with a third suspected disease, or a fourth parameter set associated with a fourth suspected disease may be designated, or fewer or more parameter sets may be designated. For example, the processor 210 may analyze a plurality of parameter sets simultaneously, according to priority, or sequentially. For example, the electrocardiogram factors (e.g., parameters) of the electrocardiogram waveform may include P wave, RR interval, R wave, QRS complex, PR interval, PR segment, ST segment, ST interval, and/or TP interval and may further include other parameters.

The processor 210 according to an embodiment may also identify the suspected disease by analyzing the ECG factors of the electrocardiogram waveform based on the stored second electrocardiogram waveform (e.g., normal electrocardiogram waveform or representative electrocardiogram waveform) previously obtained or stored and the first electrocardiogram waveform without identifying the presence or absence of an arrhythmia and/or ECG-associated symptoms. The processor 210 according to an embodiment may obtain second biometric sensing information using the second biometric sensor module 230 based on the identified suspected disease and may provide suspected disease information obtained based on the second biometric sensing information. For example, the suspected disease may include hypercalcemia, hyperkalemia, dehydration, rehydration, hyperglycemia, and hypoglycemia, and if it is a suspected disease predictable based on electrocardiogram, may further include other diseases.

Table 1 below is a table showing an example of a relationship between suspected diseases and parameters.

TABLE 1

| suspected disease | Parameter set |
|---|---|
| Hypercalcemia | Common ECG changes |
| | Shortened QT interval. |
| | Lengthened QRS duration. |
| | Bradycardia may occur. |
| | Rare ECG changes |
| | Increased QRS amplitude. |
| | Diminished T-wave amplitude |
| | Osborn-like waves. |
| | ST segment elevation in leads V1-V2. |
| | All degrees of AV block. |
| | Sinus node dysfunction and tach-brady syndrome. |
| | Ventricular tachycardia, ventricular fibrillation and torsade de pointes. |
| Hypocalcemia | Common ECG changes |
| | Lengthened QT interval (torsade de pointes is uncommon) |
| | Shortened QRS duration (has no clinical significance) |
| | Rare ECG changes |
| | AV block. |
| | Sinus bradycardia. |
| | Sinoatrial (SA) block. |
| | Ventricular fibrillation. |
| Hyperkalemia | Previously mentioned ECG changes become more pronounced. |
| | The QRS complex becomes wider. |
| Hypokalemia | T-waves become wider with lower amplitudes. T-wave inversion may |
| | occur in severe hypokalemia. |
| | ST segment depression develops and may, along with T-wave inversions, |
| | simulate ischemia. |
| | P-wave amplitude, P-wave duration and PR interval may all increase. |
| | Finally, U-waves emerge. U-waves are best seen in leads V2-V3. If |
| | the hypokalemia is severe, the U-wave may become larger than the T-wave. |
| Dehydration | Increased QRS amplitude. |
| | Augmentation of the sum of amplitudes of P waves and QRS complexes |
| | Decreased left ventricular end-systolic volume (LVESV), left ventricular |
| | end-diastolic volume (LVEDV), and stroke volume (ml) |
| Rehydration | Recovery of the P wave and ECG parameters |
| Hyperglycemia | Reduced heart rate variability |
| | Significant increments of QTC interval and PR interval |
| | Shorter mean RR intervals |
| Hypoglycemia | Prolongation of QT interval |
| | RR (distance of two nearest R points), |
| | RTC (interval from R point to peak of T wave with Bazett's correction), |
| | T wave amplitude, |
| | T wave skewness, |
| | T wave kurtosis and |
| | T wave peak-to-end |

Referring to Table 1 above, at least some of the electrocardiogram factors (parameters) may be associated with their respective suspected diseases. According to an embodiment, the processor 210 may identify the second biometric sensor module 230 for obtaining additional biometric sensing information associated with the suspected disease based on the identified suspected disease (e.g., identify the biometric sensor associated with the suspected disease among the plurality of biometric sensors) and obtain second biometric sensing information using the second biometric sensor module 230 (or from the outside). For example Table 2 below is a table illustrating an example of additional biometric sensing information (e.g., second biometric sensing information) associated with suspected diseases.

TABLE 2

| Suspected | Second biometric | |
|---|---|---|
| disease | sensing information | Means to obtain |
| Dehydration | sweat | sweat sensor |
| | blood | external electronic device (e.g., blood sensor) |
| | urine | external server (e.g., external medical information server) |
| Blood pressure | brachial blood pressure | blood flow sensor |
| | wrist blood pressure | external electronic device (e.g., blood pressure sensor) |
| | finger blood pressure | external server (e.g., external information server) |
| Blood glucose | sweat | sweat sensor |
| | blood | external electronic device (e.g., blood sensor) |
| | urine | external server (e.g., external medical information server) |
| | iris | iris sensor or external electronic device (e.g., iris sensor) |

Referring to Table 2, the processor 210 may obtain, based on the identified suspected disease, second biometric sensing information associated with the suspected disease from the second biometric sensor module 230 included in the electronic device 201 or may receive (or object) it from an external electronic device or an external server. According to an embodiment of the disclosure, the processor 210, which may be a hardware module or software module (e.g., an application program), may be a hardware component (function) or software component (program) including at least one of various sensors, data measurement module, input/output interface, a module for managing the state or environment of the electronic device 201, or communication module as included in the electronic device 201. According to an embodiment, the processor 210 may include, e.g., a hardware module, a software module, a firmware module, or a combination of two or more thereof. The processor 210 may lack at least some of the components or may include other components for performing an image processing operation in addition to the components.

The memory 240 according to an embodiment may store an application. For example, the memory 240 may store an application (function or program) related to electrocardiogram measurement, an exercise application, or a health care application. According to an embodiment, the memory 240 may store electrocardiogram measurement result information and may store a second electrocardiogram waveform that is the user's electrocardiogram waveform within a normal range based on the electrocardiogram measurement result. The memory 240 according to an embodiment may store data tables corresponding to Tables 1 and 2 above. According to an embodiment, the memory 240 may store various data generated during execution of the program 140, as well as a program (e.g., the program 140 of FIG. 1) used for functional operation. The memory 240 may include a program area 140 and a data area (not shown). The program area 140 may store relevant program information for driving the electronic device 201, such as an operating system (OS) (e.g., the OS 142 of FIG. 1) for booting the electronic device 201. The data area (not shown) may store transmitted and/or received data and generated data according to an embodiment. The memory 240 may include at least one storage medium of a flash memory, a hard disk, a multimedia card, a micro-type memory (e.g., a secure digital (SD) or an extreme digital (xD) memory), a random access memory (RAM), or a read only memory (ROM).

The display 250 according to an embodiment may display various types of information based on the control of the processor 210. For example, the display 250 may display information indicating that the electrocardiogram is normal or may display suspected disease information based on the first biometric sensing information and second biometric sensing information and arrhythmia information (presence and/or type of arrhythmia) based on the first biometric sensing information. According to an embodiment, the display 250 may be implemented as a touchscreen display. The display 250, when implemented together with an input module in the form of a touchscreen display, may display various information generated according to the user's touch. According to an embodiment, the display 250 may include at least one of a liquid crystal display (LCD), a thin film transistor LCD (TFT-LCD), an organic light emitting diode (OLED) display, a light emitting diode (LED) display, an active matrix organic LED (AMOLED) display, a micro LED display, a mini LED display, a flexible display, or a three-dimensional display. Some of the displays may be configured in a transparent type or light-transmissive type allowing the outside to be viewed therethrough. This may be configured in the form of a transparent display including a transparent OLED (TOLED) display. According to another embodiment, the electronic device 201 may further include another mounted display module (e.g., an extended display or a flexible display) in addition to the display 250.

According to an embodiment, the communication module 270 may communicate with an external electronic device (e.g., the electronic device 102 or 104 of FIG. 1, the server 108 of FIG. 1, or another user's electronic device). For example, the communication module 270 may receive, from the external electronic device, second biometric sensing information associated with the suspected disease, based on the suspected disease identified based on electrocardiogram. According to an embodiment, the communication module 270 may include a cellular module, a wireless-fidelity (Wi-Fi) module, a Bluetooth module, or a near field communication (NFC) module. Further, other modules capable of communicating with the external electronic device may be further included.

According to an embodiment, the electronic device 201 is not limited to the configuration illustrated in FIG. 2 and may further include various components.

According to an embodiment, the electronic device 201 may further include an audio module (not shown) (e.g., the audio module 170 of FIG. 1) or a vibration module (not shown) (e.g., the haptic module 179 of FIG. 1). The audio module may output sounds and may include at least one of, e.g., an audio codec, a microphone (MIC), a receiver, an earphone output (EAR_L) or a speaker. The audio module may output, as an audio signal, information related to the user's physical condition, information related to abnormal symptoms of the user's health condition, or additional information based on the obtained electrocardiogram and/or suspected disease information. For example, the vibration module may output, as vibration, information related to the user's physical condition, information related to abnormal symptoms of the user's health condition, or additional information, based on the obtained electrocardiogram and/or suspected disease information.

Major components of the electronic device 201 have been described above in connection with FIG. 2. According to an embodiment, however, all of the components of FIG. 2 are not essential components, and the electronic device 201 may be implemented with more or less components than those shown. The positions of the major components of the electronic device 201 described above in connection with FIG. 2 may be varied according to an embodiment of the disclosure.

According to various embodiments, an electronic device (e.g., the electronic device 101 of FIG. 1 or the electronic device 201 of FIG. 2) may comprise a first biometric sensor (e.g., the sensor module 176 of FIG. 1 or the first biometric sensor module 260 of FIG. 2) comprising a plurality of electrodes and a measurement sensor (e.g., the measurement module 226 of FIG. 2) electrically connected with the plurality of electrodes, a display (e.g., the display 160 of FIG. 1 or the display 250 of FIG. 2), a memory (e.g., the memory 130 of FIG. 1 or the memory 240 of FIG. 2), and a processor (e.g., the processor 120 of FIG. 1 or the processor 210 of FIG. 2) configured to obtain first biometric sensing information including a first electrocardiogram waveform using the first biometric sensor, identify a suspected disease based on the first electrocardiogram waveform and a previously obtained second electrocardiogram waveform, obtain second biometric sensing information corresponding to the identified suspected disease, and display, on the display, suspected disease information obtained based on the second biometric sensing information.

According to various embodiments, the processor may be configured to identify a presence or absence of an arrhythmia based on the first electrocardiogram waveform and, in response to identifying the presence of the arrhythmia, identify the suspected disease based on the first electrocardiogram waveform and the second electrocardiogram waveform.

According to various embodiments, the processor may be configured to identify a presence or absence of an arrhythmia based on the first electrocardiogram waveform, identify a presence or absence of a symptom associated with the first electrocardiogram waveform, and in response to identifying the presence of the arrhythmia and the presence of the symptom, identify the suspected disease based on the first electrocardiogram waveform and the second electrocardiogram waveform.

According to various embodiments, the suspected disease may include at least one of hypercalcemia, hyperkalemia, dehydration, rehydration, hyperglycemia, or hypoglycemia.

According to various embodiments, the electronic device may further comprise a second biometric sensor module. The processor may be configured to obtain the second biometric sensing information using the second biometric sensor module.

According to various embodiments, the electronic device may further comprise a communication module. The processor may be configured to obtain the second biometric sensing information from an external device through communication with the external device by using the communication module.

According to various embodiments, the processor may be configured to obtain medical information from a medical information server through communication with the medical information server by using the communication module and obtain the second biometric sensing information from the medical information.

According to various embodiments, the first electrocardiogram waveform and the second electrocardiogram waveform may include parameters respectively. The parameters may include at least one of a P wave, an RR interval, an R wave, a QRS complex, a PR interval, a PR segment, an ST segment, an ST interval, or a TP interval.

According to various embodiments, the processor may be configured to identify a first plurality of parameter sets based on the parameters of the first electrocardiogram waveform and identify a second plurality of parameter sets based on the parameters of the second electrocardiogram waveform.

According to various embodiments, the processor may be configured to compare each of the first plurality of parameter sets of the first electrocardiogram waveform with each of the second plurality of parameter sets of the second electrocardiogram waveform, identify a parameter set having a value changed by more than a specified threshold change value from a value of the parameter set of the second electrocardiogram waveform from among the first plurality of parameter sets of the first electrocardiogram waveform, and identify a suspected disease corresponding to the identified parameter set.

According to various embodiments, the first plurality of parameter sets may include at least one parameter set among a first parameter set associated with atrial fibrillation, a second parameter set associated with dehydration, a third parameter set associated with blood pressure, or a fourth parameter set associated with blood glucose.

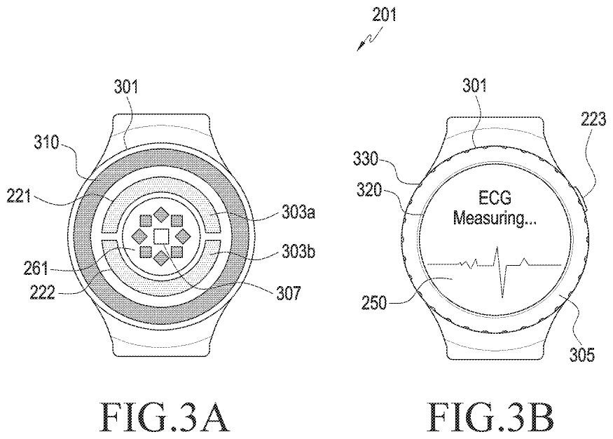

FIG. 3 A and FIG. 3B are views illustrating an example in which an electronic device is implemented as a wearable device according to an embodiment of the disclosure.

Referring to FIG. 3 A and FIG. 3B, according to an embodiment, an electronic device 201 may be, e.g., a wearable device in the form of a wrist watch, which may be worn on the user's wrist or a wearable device that may be worn on another portion of the body (e.g., head, forearm, thigh, or another body portion where electrocardiogram may be measured). The electronic device 201 according to an embodiment may include a housing 301 including a first surface 310 (e.g., rear surface), a second surface 320 (e.g., front surface), and a third surface 330 (e.g., side surface) surrounding a space between the first surface 310 (e.g., rear surface) and second surface 320 (e.g., rear surface).

Referring to FIG. 3A, a first electrode 221 and a second electrode 222 included in the electrode module 225 may be disposed on at least two portions of a first member 303a and 303b disposed on the first surface (e.g., the rear surface) of the housing 301. According to various embodiments, the first electrode 221 and the second electrode 222 may be disposed on the first surface 310 (e.g., rear surface) of the electronic device 201 to be able to contact the user's body portion (e.g., wrist) when the electronic device 201 is worn.

Referring to FIG. 3B, the electronic device 201 according to an embodiment may include the third electrode 223, included in the electrode module 220, disposed on at least a portion of the second member 305 formed to surround the display 250 disposed on the second surface 320 (e.g., the front surface) that is another surface of the housing 301.

According to various embodiments, when the electronic device 201 is worn, the third electrode 223 may be disposed on at least one portion of the housing 301 so as not to come into contact with the user's body portion. According to an embodiment, the third electrode 223 may be disposed on the second surface 320 (e.g., the front surface) of the electronic device 201. For example, the third electrode 223 may be disposed on or included in the display 250 in the form of a transparent electrode (e.g., indium tin oxide, ITO). According to some embodiments, there may be a plurality of third electrodes 223. The plurality of third electrodes 223 may operate as one channel or operate as different channels. The electronic device 201 may include at least one sensor 261 disposed on a third member 307, which is surrounded by the first member 303a and 303b disposed on the first surface, to be positioned in contact or proximity to the body skin. The at least one sensor 261 may be included in the sensor module 260. For example, the at least one sensor 261 may be a sensor capable of measuring at least one biometric signal. For example, the third member 307 may include at least one light source (e.g., an infrared LED) irradiating light to the skin, and the at least one sensor 261 may include at least one photodetector. For example, the third electrode 223 may be disposed on a third surface 330 (e.g., a side surface) which is another surface of the housing 301. According to an embodiment, the third electrode 223 may have a button shape disposed on a side surface of the electronic device 201.

The processor 210 according to an embodiment may identify that at least some of the plurality of electrodes (e.g., the first electrode 221, the second electrode 222, and the third electrode 223) are in contact with a portion of the human body. The processor 210 may control the measurement module 230 to operate in an electrocardiogram measurement mode, based on contact of the third electrode 223 to a second portion of the human body (e.g., a finger of the other hand or a connection point of a finger of the other hand), in a state in which the first electrode 221 and the second electrode 222 contact a first portion of the human body (e.g., a wrist portion) or connection points (not shown) of the first portion (e.g., a first connection point and a second connection point).

According to an embodiment, in FIG. 3, the electrode module 225 includes three electrodes. However, the number of electrodes may be fewer than three, e.g., two, or more than three.

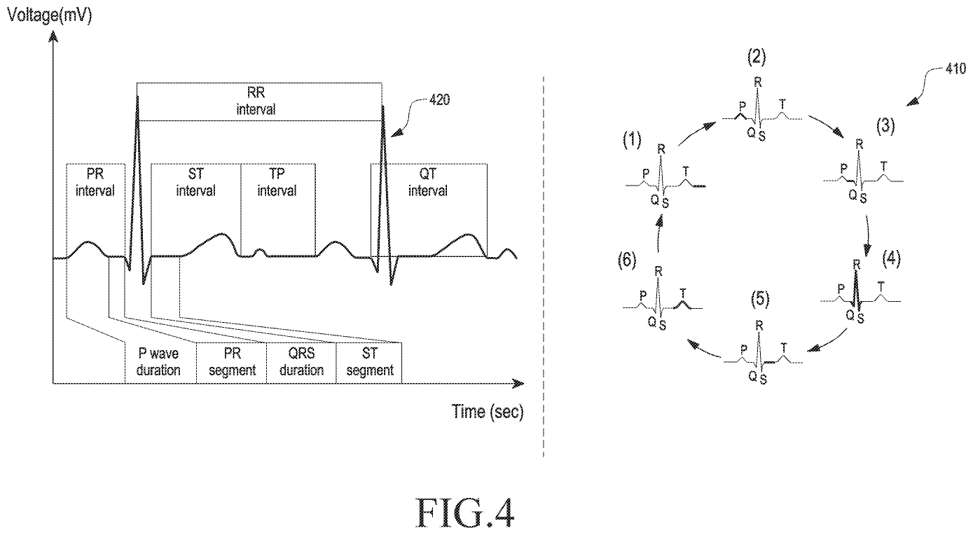

FIG. 4 is a view illustrating an electrocardiogram waveform based on heartbeat cycle according to an embodiment of the disclosure.

Referring to FIG. 4, according to an embodiment, reference numeral 410 may denote the heartbeat cycle, and reference numeral 420 may denote the electrocardiogram waveform according to the heartbeat cycle.

The heartbeat cycle 410 according to an embodiment may include (1) a process in which the ventricles are relaxed and the ventricles are repolarized so that blood enters the ventricles, (2) a process of contraction of the left and right atrial walls, (3) a process of conduction of excitation of the atria and the ventricles, (4) a process of complex excitation of the left and right ventricular walls and ventricular septum, (5) a process of ventricular excitation, and/or (6) a process in which the excited ventricular wall is restored.

The electrocardiogram waveform 420 based on the heartbeat cycle 410 according to an embodiment may include electrocardiogram factors corresponding to processes (1) to (6). The ECG waveform 420 according to an embodiment may be a waveform of voltage (mV) according to the time (sec) measured in the user's body using a plurality of electrodes (e.g., at least two electrodes). For example, the electrocardiogram factors of the electrocardiogram waveform 420 may include P wave, RR interval, R wave, QRS complex, PR interval, PR segment, ST segment, ST interval, TP interval, and/or QT interval. The electrocardiogram factor corresponding to process (1) may be TP interval, the electrocardiogram factor corresponding to process (2) may be P wave, the electrocardiogram factor corresponding to process (3) may be PR interval, the electrocardiogram factor corresponding to process (4) may be QRS complex, the electrocardiogram factor corresponding to process (5) may be ST segment, and the electrocardiogram factor corresponding to process (6) may be T wave.

The processor 210 according to an embodiment may identify the presence and type of arrhythmia based on the first parameter set (e.g., the presence or absence of the P wave and/or regularity of the RR interval) among the electrocardiogram factors (e.g., parameters) of the electrocardiogram waveform 420. The processor 210 according to an embodiment may compare at least some (e.g., at least some parameter sets among the plurality of parameter sets) of the ECG factors of the measured first ECG waveform and at least one some (e.g., at least some parameter sets among the plurality of parameter sets) of the ECG factors of the previously obtained second ECG waveform (e.g., the normal ECG waveform) and identify the suspected disease based on at least one ECG factor for which a difference by a designated threshold or more is made therebetween. For example, the suspected disease may include hypercalcemia, hyperkalemia, dehydration, rehydration, hyperglycemia, and/or hypoglycemia.

For example, the processor 210 may compare the first ST interval and first QRS interval of the measured first ECG waveform and the second ST interval and second QRS interval of the previously obtained second ECG waveform (e.g., normal ECG waveform) and, if the difference between the first ST interval and the second ST interval and the difference between the first QRS interval and the second QRS interval are a designated threshold variation or more, identify that the suspected disease is hypoglycemia and obtain blood glucose sensing information (e.g., blood glucose level), as the second biometric sensing information, through an external electronic device or a blood glucose meter which is an additional second biometric sensor associated with hypoglycemia. As another example, the processor 210 may compare the first QRS complex of the measured first electrocardiogram waveform with the second QRS complex of the previously obtained second electrocardiogram waveform (e.g., normal electrocardiogram waveform) and, if the difference between the first QRS complex and the second QRS complex is a designated threshold variation or more, identify that the suspected disease is hyperkalemia and obtain blood sensing information (e.g., blood potassium ion concentration), as the second biometric sensing information, through an external electronic device or a blood sensor which is an additional second biometric sensor associated with hyperkalemia. The processor 210 may identify another suspected disease by comparing other ECG factors (parameter sets) between the measured first ECG waveform and the previously obtained second ECG waveform (e.g., normal ECG waveform) and obtain other second biometric sensing information through an external electronic device or an additional second biometric sensor associated with the suspected disease.

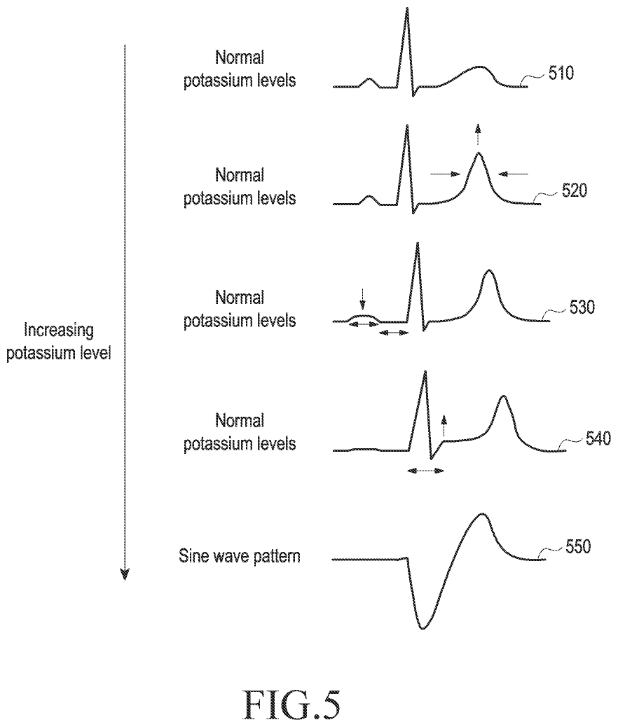

FIG. 5 is a view illustrating a change in electrocardiogram waveform due to an increase in blood potassium concentration according to an embodiment of the disclosure.

Referring to FIG. 5, the processor 210 according to an embodiment may obtain a plurality of ECG waveforms for the user periodically or with a designated time interval or a time difference and may identify changes between the plurality of electrocardiogram waveforms. When a first ECG waveform 510 to a fifth ECG waveform 550 are obtained, the processor 210 according to an embodiment may compare the plurality of parameter sets for the first ECG waveform 510 to the fifth ECG waveform 550 to identify a change in the plurality of parameter sets. When the change in the plurality of parameter sets is a change corresponding to an increase in the suspected disease (e.g., blood potassium concentration (potassium level)), the processor 210 according to an embodiment may identify an internal blood sensor, as the second biometric sensor to measure the blood potassium concentration, operate the blood sensor to obtain blood sensing information or obtain blood sensing information from an external blood sensor or an external server. The processor 210 according to an embodiment may provide information regarding the suspected disease (e.g., hyperkalemia) based on the obtained blood sensing information. According to an embodiment, the processor 210 may provide prevention information or treatment information corresponding to the suspected disease information together with the suspected disease information.

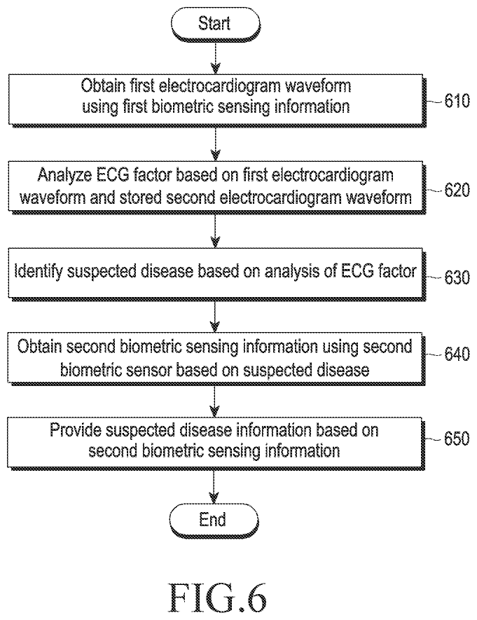

FIG. 6 is a flowchart illustrating an operation of providing electrocardiogram-based health information in an electronic device according to an embodiment of the disclosure.

Referring to FIG. 6, according to an embodiment, a processor (e.g., the processor 120 of FIG. 1 or the processor 210 of FIG. 2) of an electronic device (e.g., the electronic device 101 of FIG. 1 or the electronic device 201 of FIG. 2) may perform at least one of operations 610 to 650.

In operation 610, the processor 210 according to an embodiment may obtain (or measure) a first ECG waveform using a first biometric sensor module 220 (e.g., an ECG sensor). For example, the processor 210 may store the first ECG waveform in the memory 240.

In operation 620, the processor 210 according to an embodiment may analyze the ECG factors (e.g., parameters) of the ECG waveform based on the first ECG waveform and a previously obtained or stored second ECG waveform (e.g., a normal ECG waveform). For example, the parameters may include a plurality of feature points (e.g., P-wave, QRS complex, T-wave) or sections associated with a plurality of feature points (e.g., segments or duration associated with P-wave, QRS complex, and/or T-wave). For example, the processor 210 may analyze all of the parameters or a parameter set obtained by combining at least some parameters from all of the parameters. For example, the parameter set may be designated as a parameter set associated with a suspected disease for each suspected disease that may be suspected in the ECG waveform. For example, a first parameter set associated with a first suspected disease, a second parameter set associated with a second suspected disease, a third parameter set associated with a third suspected disease, or a fourth parameter set associated with a fourth suspected disease may be designated, or fewer or more parameter sets may be designated. For example, the processor 210 may analyze a plurality of parameter sets simultaneously, according to priority, or sequentially. For example, the electrocardiogram factors (e.g., parameters) of the electrocardiogram waveform may include P wave, RR interval, R wave, QRS complex, PR interval, PR segment, ST segment, ST interval, and/or TP interval and may further include other parameters.

For example, the processor 210 may compare first ECG factors (e.g., at least some parameter sets among the plurality of parameter sets) of the measured first ECG waveform and second ECG factors (e.g., at least some parameter sets among the plurality of parameter sets) of the second ECG waveform (e.g., the normal ECG waveform) and identify an ECG factor (e.g., parameter set) which is changed by a designated threshold variation or more. For example, the processor 210 may identify an ECG factor which is changed by the designated threshold variation or more among P wave, RR interval, R wave, QRS complex, PR interval, PR segment, ST segment, ST interval, and/or TP interval.

In operation 630, the processor 210 according to an embodiment may identify the suspected disease based on the analysis of electrocardiogram factors (parameters). For example, the processor 210 may identify the related suspected disease based on the ECG factor (at least one parameter set) identified as changed by the designated threshold variation or more. For example, the suspected disease may include hypercalcemia, hyperkalemia, dehydration, rehydration, hyperglycemia, and/or hypoglycemia. For example, the processor 210 may compare the first ST interval and first QRS interval of the first ECG waveform and the second ST interval and second QRS interval of the previously obtained second ECG waveform (e.g., normal ECG waveform) and, if the difference between the first ST interval and the second ST interval and the difference between the first QRS interval and the second QRS interval are a designated threshold variation or more, identify that the suspected disease is hypoglycemia. As another example, the processor 210 may compare the first QRS complex of the first electrocardiogram waveform with the second QRS complex of the previously obtained second electrocardiogram waveform (e.g., normal electrocardiogram waveform) and, if the difference between the first QRS complex and the second QRS complex is a designated threshold variation or more, identify that the suspected disease is hyperkalemia. Further, the processor 210 may compare the measured first ECG waveform and the previously obtained second ECG waveform (e.g., normal ECG waveform), identify another parameter set for which the difference is a designated threshold variation or more, and identify the suspected disease corresponding to the identified other parameter set.

In operation 640, the processor 210 according to an embodiment may obtain second biometric sensing information using the second biometric sensor module (e.g., 230) based on the identified suspected disease. For example, when hypoglycemia is identified as the suspected disease, the processor 210 may obtain blood sensing information (e.g., blood glucose level), as second biometric sensing information, through a blood glucose meter included in an additional second biometric sensor module 230 associated with hypoglycemia or an external electronic device. As another example, when hyperkalemia is identified as the suspected disease, the processor 210 may obtain blood sensing information (e.g., blood potassium ion concentration), as second biometric sensing information, through a blood sensor included in an additional second biometric sensor module 230 associated with hyperkalemia or an external electronic device. The processor 210 may further obtain other second biometric sensing information through an additional second biometric sensor module 230 associated with another suspected disease or an external electronic device.

In operation 650, the processor 210 according to an embodiment may provide suspected disease information based on the second biometric sensing information. For example, the processor 210 may provide the suspected disease information, as health information based on electrocardiogram, to the display 250. For example, when the blood glucose level obtained as the second biometric sensing information based on ECG corresponds to a high blood glucose level, the processor 210 may provide information indicating a high blood glucose level. As another example, when the blood potassium ion concentration obtained as the second biometric sensing information based on ECG corresponds to hyperkalemia, the processor 210 may provide information indicating hyperkalemia. In addition thereto, the processor 210 may further provide other suspected disease information corresponding to another value obtained as other second biometric sensing information based on ECG.

According to various embodiments, a method for providing electrocardiogram-based health information in an electronic device (e.g., the electronic device 101 of FIG. 1 or the electronic device 201 of FIG. 2) may comprise obtaining first biometric sensing information comprising a first electrocardiogram waveform using a first biometric sensor comprising a plurality of electrode sand a measurement sensor, identifying a suspected disease based on the first electrocardiogram waveform and a previously obtained second electrocardiogram waveform, obtaining second biometric sensing information corresponding to the identified suspected disease, and displaying, on a display, suspected disease information obtained based on the second biometric sensing information.

According to various embodiments, the method may further comprise identifying a presence or absence of an arrhythmia based on the first electrocardiogram waveform and identifying the suspected disease based on the first electrocardiogram waveform and the second electrocardiogram waveform in response to identifying the presence of the arrhythmia.

According to various embodiments, the method may further comprise identifying a presence or absence of a symptom associated with the first electrocardiogram waveform, in response to identifying the presence of the arrhythmia and the presence of the symptom, identifying the suspected disease based on the first electrocardiogram waveform and the second electrocardiogram waveform.

According to various embodiments, the suspected disease may include one of hypercalcemia, hyperkalemia, dehydration, rehydration, hyperglycemia, or hypoglycemia.

According to various embodiments, the method may obtain the second biometric sensing information using a second biometric sensor module of the electronic device.

According to various embodiments, the method may further comprise obtaining the second biometric sensing information through communication with an external medical information server or an external device by using the communication module.

According to various embodiments, in the method, the first electrocardiogram waveform and the second electrocardiogram waveform include parameters respectively may include parameters. The parameters may include at least one of a P wave, an RR interval, an R wave, a QRS complex, a PR interval, a PR segment, an ST segment, an ST interval, or a TP interval. The method may further comprise a first plurality of parameter sets based on the parameters of the first electrocardiogram waveform and identify a second plurality of parameter sets based on the parameters of the second electrocardiogram waveform.

According to various embodiments, the method may further comprise comparing each of the first plurality of parameter sets of the first electrocardiogram waveform with each of the second plurality of parameter sets of the second electrocardiogram waveform, identifying a parameter set having a value changed by more than a specified threshold change value from a value of the parameter set of the second electrocardiogram waveform from among the first plurality of parameter sets of the first electrocardiogram waveform.

According to various embodiments, the first plurality of parameter sets may include at least one parameter set among a first parameter set associated with atrial fibrillation, a second parameter set associated with dehydration, a third parameter set associated with blood pressure, or a fourth parameter set associated with blood glucose.

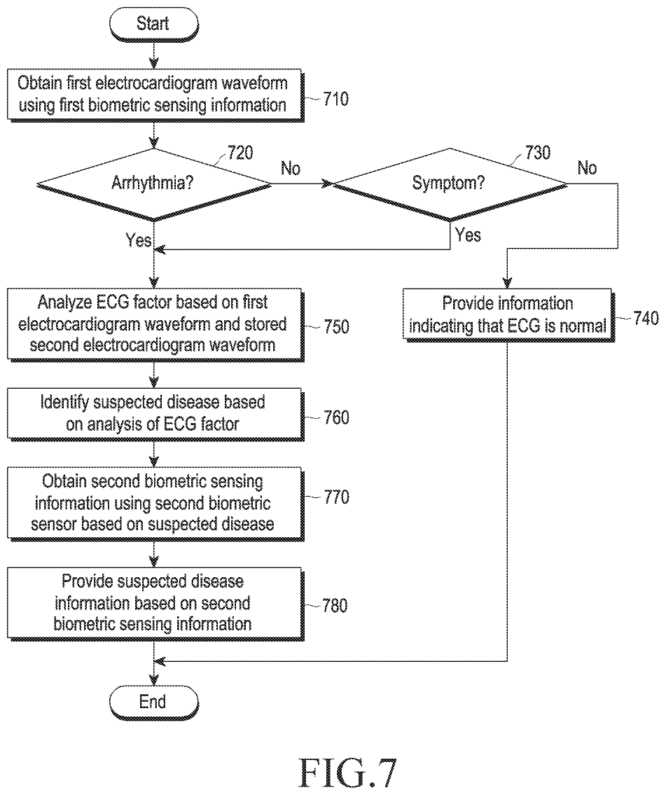

FIG. 7 is a flowchart illustrating an operation of providing health information based on arrhythmia in an electronic device according to an embodiment of the disclosure.

Referring to FIG. 7, according to an embodiment, a processor (e.g., the processor 120 of FIG. 1 or the processor 210 of FIG. 2) of an electronic device (e.g., the electronic device 101 of FIG. 1 or the electronic device 201 of FIG. 2) may perform at least one of operations 710 to 780.

In operation 710, the processor 210 according to an embodiment may obtain (or measure) a first ECG waveform using a first biometric sensor module (e.g., a first biometric sensor or ECG sensor). For example, the processor 210 may store the first ECG waveform in the memory 240.

In operation 720, the processor 210 according to an embodiment may identify whether an arrhythmia (e.g., atrial fibrillation) is present based on the first electrocardiogram waveform. The processor 210 according to an embodiment may identify the presence and type of arrhythmia based on the presence or absence of the P wave and/or regularity of the RR interval among the electrocardiogram factors of the electrocardiogram waveform.

In operation 730, when an arrhythmia is not identified, the processor 210 according to an embodiment may identify whether an ECG-associated symptom present. For example, the processor 210 may display at least one symptom check item for identifying whether there is an ECG-associated symptom on the display 250 and may identify the presence or absence of an ECG-associated symptom based on the symptom checked by user input. For example, electrocardiogram-associated symptoms may include rapid heartbeat, skipping heartbeats, fatigue, shortness of breath, chest pain, tightness, fainting, and/or dizziness. For example, symptom information input by the user may be stored, and the stored symptom information may be transmitted to the outside to be shared with others or medical staff associated with the user.

In operation 740, the processor 210 according to an embodiment may provide information indicating that the electrocardiogram is normal when an arrhythmia is not identified and there are no electrocardiogram-associated symptoms. For example, the processor 210 may provide information indicating that ECG is normal, as health information based on electrocardiogram, to the display 250.

In operation 750, if there is an ECG-associated symptom in a state in which an arrhythmia is not identified as absent or is identified as present, the processor 210 according to an embodiment may analyze the ECG factors (e.g., parameters) of the ECG waveform based on the first ECG waveform and a previously obtained or stored second ECG waveform (e.g., a normal ECG waveform). For example, the parameters may include a plurality of feature points (e.g., P-wave, QRS complex, T-wave) or sections associated with a plurality of feature points (e.g., segments or duration associated with P-wave, QRS complex, and/or T-wave). For example, the processor 210 may analyze all of the parameters or a parameter set obtained by combining at least some parameters from all of the parameters. For example, the parameter set may be designated as a parameter set associated with a suspected disease for each suspected disease that may be suspected in the ECG waveform. For example, a first parameter set associated with a first suspected disease, a second parameter set associated with a second suspected disease, a third parameter set associated with a third suspected disease, or a fourth parameter set associated with a fourth suspected disease may be designated, or fewer or more parameter sets may be designated. For example, the processor 210 may analyze a plurality of parameter sets simultaneously, according to priority, or sequentially. For example, the electrocardiogram factors (e.g., parameters) of the electrocardiogram waveform may include P wave, RR interval, R wave, QRS complex, PR interval, PR segment, ST segment, ST interval, and/or TP interval and may further include other parameters.

For example, the processor 210 may compare first ECG factors (e.g., at least some parameter sets among the plurality of parameter sets) of the measured first ECG waveform and second ECG factors (e.g., at least some parameter sets among the plurality of parameter sets) of the second ECG waveform (e.g., the normal ECG waveform) and identify an ECG factor (e.g., parameter set) which is changed by a designated threshold variation or more. For example, the processor 210 may identify an ECG factor which is changed by the designated threshold variation or more among P wave, RR interval, R wave, QRS complex, PR interval, PR segment, ST segment, ST interval, and/or TP interval.

In operation 760, the processor 210 according to an embodiment may identify the suspected disease based on the analysis of electrocardiogram factors. For example, the processor 210 may identify the suspected disease based on the ECG factor (e.g., parameter set) identified as changed by the designated threshold variation or more. For example, the suspected disease may include hypercalcemia, hyperkalemia, dehydration, rehydration, hyperglycemia, and/or hypoglycemia. For example, the processor 210 may compare the first ST interval and first QRS interval of the first ECG waveform and the second ST interval and second QRS interval of the previously obtained second ECG waveform (e.g., normal ECG waveform) and, if the difference between the first ST interval and the second ST interval and the difference between the first QRS interval and the second QRS interval are a designated threshold variation or more, identify that the suspected disease is hypoglycemia. As another example, the processor 210 may compare the first QRS complex of the first electrocardiogram waveform with the second QRS complex of the previously obtained second electrocardiogram waveform (e.g., normal electrocardiogram waveform) and, if the difference between the first QRS complex and the second QRS complex is a designated threshold variation or more, identify that the suspected disease is hyperkalemia. Further, the processor 210 may compare the measured first ECG waveform and the previously obtained second ECG waveform (e.g., normal ECG waveform), identify another parameter set for which the difference is a designated threshold variation or more, and identify the suspected disease corresponding to the identified other parameter set.

In operation 770, the processor 210 according to an embodiment may obtain second biometric sensing information using the second biometric sensor module 230 (e.g., a second biometric sensor) based on the identified suspected disease. For example, when hypoglycemia is identified as the suspected disease, the processor 210 may obtain the blood glucose level, as second biometric sensing information, through a blood glucose meter, which is an additional second biometric sensor associated with hypoglycemia, or an external electronic device. As another example, when hyperkalemia is identified as the suspected disease, the processor 210 may obtain the blood potassium ion concentration, as second biometric sensing information, through a blood sensor, which is an additional second biometric sensor associated with hyperkalemia, or an external electronic device. The processor 210 may further obtain other second biometric sensing information through an additional second biometric sensor associated with another suspected disease or an external electronic device.

In operation 780, the processor 210 according to an embodiment may provide suspected disease information based on the second biometric sensing information. For example, the processor 210 may provide the suspected disease information, as health information based on electrocardiogram, to the display 250. For example, when the blood glucose level obtained as the second biometric sensing information based on ECG corresponds to a high blood glucose level, the processor 210 may provide information indicating a high blood glucose level. As another example, when the blood potassium ion concentration obtained as the second biometric sensing information based on ECG corresponds to hyperkalemia, the processor 210 may provide information indicating hyperkalemia. In addition thereto, the processor 210 may further provide other disease information corresponding to another value obtained as other second biometric sensing information based on ECG.

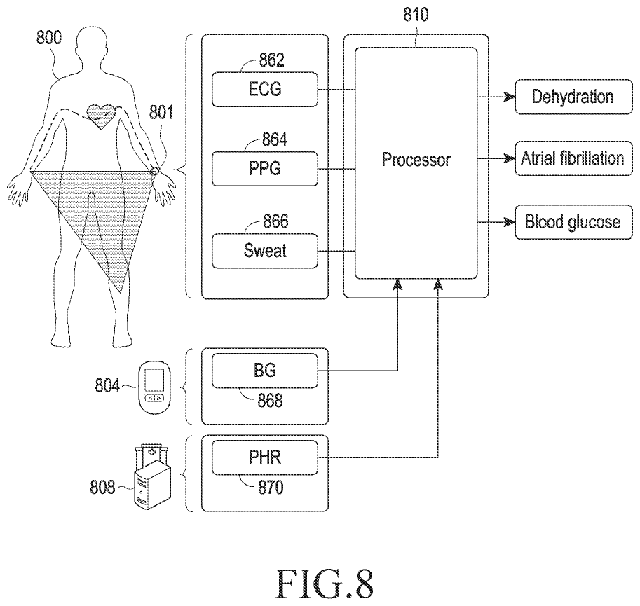

FIG. 8 is a view illustrating an example of providing suspected disease information using biometric sensing information obtained from a sensor module included in an electronic device, an external electronic device, and an external server, in the electronic device according to an embodiment of the disclosure.

Referring to FIG. 8, an electronic device 801 (e.g., the electronic device 101 of FIG. 1 or the electronic device 201 of FIG. 2) according to an embodiment may include an ECG sensor 862, a PPG sensor 864, a SWEAT sensor 866, a communication module (not shown), and a processor 810, and may communicate with an external electronic device 804 (e.g., the electronic device 104 of FIG. 1) and an external server 808 (e.g., the server 108 of FIG. 1) through the communication module.

The ECG sensor 862, the PPG sensor 864, and the SWEAT sensor 866 according to an embodiment may be configured individually or in combination, or some sensors of the ECG sensor 862, the PPG sensor 864, the SWEAT sensor 866 may be integrated.

The ECG sensor 862 according to an embodiment may include an electrode module including a plurality of electrodes (e.g., at least two electrodes) that may contact the user's body and an electrocardiogram measurement module (or a processing module) and may measure the electrocardiogram through the electrode module and the electrocardiogram module. The PPG sensor 864 according to an embodiment may include an electrode module including a plurality of electrodes (e.g., at least two electrodes) that may contact the user's body and a pulse wave measurement module (or a processing module) and may measure the pulse wave through the electrode module and the pulse wave measurement module. The SWEAT sensor 866 may include an electrode module including a plurality of electrodes (e.g., at least two electrodes) that may contact the user's body and a hydration and/or dehydration measurement module (or a processing module) and measure the degree of hydration and/or dehydration through the electrode module and the hydration and/or dehydration measurement module. According to an embodiment, some of the ECG sensor 862, the PPG sensor 864, and the SWEAT sensor 866 may share one electrode module through a switching circuit or may have and use separate electrode modules.

The processor 810 according to an embodiment may obtain ECG sensing information, PPG sensing information, and SWEAT sensing information measured for the user 800 from the ECG sensor 862, the PPG sensor 864, and the SWEAT sensor 866, respectively. The processor 810 according to an embodiment may receive BG sensing information measured for the user 800 from the blood glucose sensor 868 of the external electronic device 804 and may receive medical record information (personal health record (PHR)) 870 for the user 800 from the external server 808.

The processor 810 according to an embodiment may drive (by instructions) a processing engine (e.g., an arrhythmia engine (e.g., arrhythmia identification algorithm or atrial fibrillation identification algorithm), a dehydration engine (e.g., a dehydration identification algorithm), and/or a blood glucose engine (e.g., a blood glucose level identification algorithm)) stored in the memory 240 to process ECG sensing information, PPG sensing information, and/or SWEAT sensing information. The processor 810 according to an embodiment may drive (by instructions) a learning engine (or learning algorithm) for learning the ECG sensing information, PPG sensing information, SWEAT sensing information, BG sensing information and/or medical record information stored in the memory (e.g., 240) to perform learning and obtain ECG sensing information, PPG sensing information, SWEAT sensing information, BG sensing information, and/or medical record information according to the result of learning. For example, the biometric signal obtained by the electrode module of the ECG sensor 862, the PPG sensor 864, and the SWEAT sensor 866 may be converted into digitized biometric signal data through an analog digital converter (ADC) of the measurement module and transferred to the processor 810. The processor 810 may process the ECG sensing information, PPG sensing information, and/or SWEAT sensing information using the biometric signal data and the processing engine.

The processor 810 according to an embodiment may identify the presence or absence of atrial fibrillation (e.g., arrhythmia) using the arrhythmia engine and the ECG sensing information (e.g., the first biometric sensing information) and, if atrial fibrillation is identified, output information indicating atrial fibrillation. The processor 810 according to an embodiment may identify the presence or absence of dehydration using the dehydration engine and the ECG sensing information (e.g., the first biometric sensing information), the PPG sensing information, and the BG sensing information and, if dehydration is identified, output information indicating dehydration. The processor 810 according to an embodiment may identify blood glucose using the blood glucose engine, dehydration engine, ECG sensing information (e.g., first biometric sensing information), PPG sensing information, and BG sensing information and output blood glucose information. As described above, according to FIG. 8, the electronic device 801 may obtain more accurate suspected disease information by using information from the external device 804 (e.g., an external biometric sensor) or the external server 808 as well as the biometric sensors in the electronic device 801. For example, the electronic device 801 may correct the biometric sensing information obtained by the biometric sensors in the electronic device 801, using the biometric sensing information obtained through the external biometric sensor and increase the accuracy of biometric sensing information processing by using the corrected biometric sensing information. For example, the electronic device 801 may increase the accuracy of biometric sensing information processing by using the PHR data received from an external medical center (e.g., measurement history information about the user of the electronic device 801, gender, age, and/or demographic information), together with the biometric sensing information obtained by the biometric sensors in the electronic device 801.

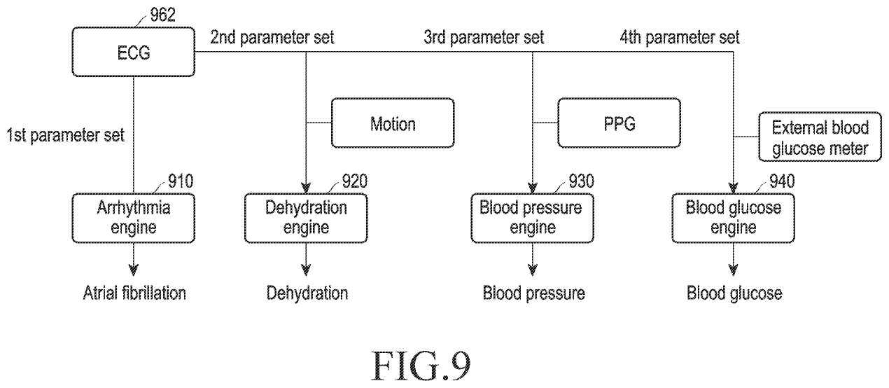

FIG. 9 is a view illustrating an example of an operation of identifying suspected disease information by a plurality of parameter sets based on electrocardiogram in an electronic device according to an embodiment of the disclosure.

Referring to FIG. 9, according to an embodiment, a processor (e.g., the processor 120 of FIG. 1 or the processor 210 of FIG. 2) of an electronic device (e.g., the electronic device 101 of FIG. 1 or the electronic device 201 of FIG. 2) may obtain a plurality of different parameter sets for individually identifying a plurality of suspected diseases from a first ECG waveform obtained using an ECG sensor 962 (e.g., the first sensor module 220 of FIG. 2 or the ECG sensor 862 of FIG. 8) and identify the presence or absence of each of the plurality of suspected diseases using each of the plurality of parameters.

According to an embodiment, when an arrhythmia and/or symptom is identified based on the first ECG waveform, the processor 210 may obtain a plurality of different parameter sets for identifying each of the plurality of suspected diseases from the first ECG waveform. According to an embodiment, the plurality of parameter sets may be obtained simultaneously, according to priority, or sequentially. According to an embodiment, the processor 210 may obtain a first parameter set associated with a first suspected disease, a second parameter set associated with a second suspected disease, a third parameter set associated with a third suspected disease, and a fourth parameter set associated with a fourth suspected disease based on the first electrocardiogram waveform. According to an embodiment, some parameters of the parameter sets may overlap each other.

According to an embodiment, the processor 210 may identify the presence or absence of atrial fibrillation using (or by analyzing) the first parameter set through the arrhythmia engine 910 when obtaining the first parameter associated with the first suspected disease.

According to an embodiment, the processor 210 may further obtain motion sensing information, as the second biometric sensing information, which is additional biometric sensing information required for identifying the second suspected disease, using a motion sensor (motion) (e.g., the second sensing module 230 of FIG. 2), when obtaining the second parameter associated with the second suspected disease and may identify the presence or absence of dehydration using (or by analyzing) the second parameter and motion sensing information through the dehydration engine 920.

According to an embodiment, the processor 210 may further obtain PPG sensing information, as the second biometric sensing information, which is additional biometric sensing information required for identifying the third suspected disease, using a PPG sensor (PPG) (e.g., the second sensing module 230 of FIG. 3), when obtaining the third parameter associated with the third suspected disease and may identify a high blood pressure or low blood pressure using (or by analyzing) the third parameter and PPG sensing information through the blood pressure engine 930.

According to an embodiment, the processor 210 may further obtain blood glucose sensing information, as the second biometric sensing information, which is additional biometric sensing information required for identifying the fourth suspected disease, using an external blood glucose meter (e.g., the external electronic device 804 of FIG. 8), when obtaining the fourth parameter associated with the fourth suspected disease and may identify a high or low blood glucose level using (or by analyzing) the fourth parameter and blood glucose sensing information through the blood glucose engine 940.

According to an embodiment, the arrhythmia engine 910, the dehydration engine 920, the blood pressure engine 930, and the blood glucose engine 940 may be software modules (or algorithms) stored in a memory (e.g., the memory 240 of FIG. 2) and executed through instructions by the processor 210. Alternatively, the arrhythmia engine 910, the dehydration engine 920, the blood pressure engine 930, and the blood glucose engine 940 may be hardware modules included in, or provided independently from, the processor 210.

According to an embodiment, the processor 210 may obtain fewer or more parameter sets than those of the first to fourth parameter sets and may more or less identify other various suspected diseases depending on the number of parameter sets obtained. For example, the suspected disease may be various diseases that may be suspected from the electrocardiogram waveform and may include other suspected diseases in addition to the suspected diseases disclosed herein.

The electronic device according to various embodiments may be one of various types of electronic devices. The electronic devices may include, for example, a portable communication device (e.g., a smart phone), a computer device, a portable multimedia device, a portable medical device, a camera, a wearable device, or a home appliance. According to an embodiment of the disclosure, the electronic devices are not limited to those described above.

It should be appreciated that various embodiments of the disclosure and the terms used therein are not intended to limit the technological features set forth herein to particular embodiments and include various changes, equivalents, or replacements for a corresponding embodiment. With regard to the description of the drawings, similar reference numerals may be used to refer to similar or related elements. It is to be understood that a singular form of a noun corresponding to an item may include one or more of the things, unless the relevant context clearly indicates otherwise. As used herein, each of such phrases as “A or B,” “at least one of A and B,” “at least one of A or B,” “A, B, or C,” “at least one of A, B, and C,” and “at least one of A, B, or C,” may include all possible combinations of the items enumerated together in a corresponding one of the phrases. As used herein, such terms as “1st” and “2nd,” or “first” and “second” may be used to simply distinguish a corresponding component from another, and does not limit the components in other aspect (e.g., importance or order). It is to be understood that if an element (e.g., a first element) is referred to, with or without the term “operatively” or “communicatively”, as “coupled with,” “coupled to,” “connected with,” or “connected to” another element (e.g., a second element), it means that the element may be coupled with the other element directly (e.g., wiredly), wirelessly, or via a third element.

As used herein, the term “module” may include a unit implemented in hardware, software, or firmware, and may interchangeably be used with other terms, for example, “logic,” “logic block,” “part,” or “circuitry”. A module may be a single integral component, or a minimum unit or part thereof, adapted to perform one or more functions. For example, according to an embodiment, the module may be implemented in a form of an application-specific integrated circuit (ASIC).

Various embodiments as set forth herein may be implemented as software (e.g., the program 140) including one or more instructions that are stored in a storage medium (e.g., internal memory 136 or external memory 138) that is readable by a machine (e.g., the electronic device 101). For example, a processor (e.g., the processor 120) of the machine (e.g., the electronic device 101) may invoke at least one of the one or more instructions stored in the storage medium, and execute it, with or without using one or more other components under the control of the processor. This allows the machine to be operated to perform at least one function according to the at least one instruction invoked. The one or more instructions may include a code generated by a complier or a code executable by an interpreter. The machine-readable storage medium may be provided in the form of a non-transitory storage medium. Wherein, the term “non-transitory” simply means that the storage medium is a tangible device, and does not include a signal (e.g., an electromagnetic wave), but this term does not differentiate between where data is semi-permanently stored in the storage medium and where the data is temporarily stored in the storage medium.

According to an embodiment, a method according to various embodiments of the disclosure may be included and provided in a computer program product. The computer program products may be traded as commodities between sellers and buyers. The computer program product may be distributed in the form of a machine-readable storage medium (e.g., compact disc read only memory (CD-ROM)), or be distributed (e.g., downloaded or uploaded) online via an application store (e.g., Play Store™), or between two user devices (e.g., smart phones) directly. If distributed online, at least part of the computer program product may be temporarily generated or at least temporarily stored in the machine-readable storage medium, such as memory of the manufacturer's server, a server of the application store, or a relay server.

According to various embodiments, each component (e.g., a module or a program) of the above-described components may include a single entity or multiple entities. According to various embodiments, one or more of the above-described components may be omitted, or one or more other components may be added. Alternatively or additionally, a plurality of components (e.g., modules or programs) may be integrated into a single component. In such a case, according to various embodiments, the integrated component may still perform one or more functions of each of the plurality of components in the same or similar manner as they are performed by a corresponding one of the plurality of components before the integration. According to various embodiments, operations performed by the module, the program, or another component may be carried out sequentially, in parallel, repeatedly, or heuristically, or one or more of the operations may be executed in a different order or omitted, or one or more other operations may be added.

According to various embodiments, there may be provided a non-volatile storage medium storing instructions, wherein the instructions are configured to, when executed by an electronic device, cause the electronic device to perform at least one operation, the at least one operation comprising obtaining first biometric sensing information comprising a first electrocardiogram waveform using a first biometric sensor comprising a plurality of electrodes and a measurement sensor, identifying a suspected disease based on the first electrocardiogram waveform and a previously obtained second electrocardiogram waveform, obtaining second biometric sensing information corresponding to the identified suspected disease, and displaying, on a display, suspected disease information obtained based on the second biometric sensing information.

According to various embodiments, wherein the instructions are configured to, when executed by the electronic device, cause the electronic device to perform the at least one operation, the at least one operation comprising providing another suspected disease information corresponding to another value obtained as other second biometric sensing information based on previously obtained second electrocardiogram waveform.

According to various embodiments, wherein, when a blood glucose level obtained as the second biometric sensing information based on the previously obtained second electrocardiogram waveform corresponds to a high blood glucose level, display information indicating the high blood glucose level.

According to various embodiments, when a blood potassium ion concentration obtained as the second biometric sensing information based on previously obtained second electrocardiogram waveform corresponds to hyperkalemia, display information indicating the hyperkalemia.

While the disclosure has been shown and described with reference to various embodiments thereof, it will be understood by those skilled in the art that various changes in form and details may be made therein without departing from the spirit and scope of the disclosure as defined by the appended claims and their equivalents.

1. An electronic device comprising:
a first biometric sensor comprising a plurality of electrodes and a measurement sensor electrically connected with the plurality of electrodes;
a display;
a memory; and
a processor configured to:
obtain first biometric sensing information including a first electrocardiogram waveform using the first biometric sensor,
identify a suspected disease based on the first electrocardiogram waveform and a previously obtained second electrocardiogram waveform,
obtain second biometric sensing information corresponding to the identified suspected disease, and
display, on the display, suspected disease information obtained based on the second biometric sensing information.

2. The electronic device of claim 1, wherein the processor is further configured to:
identify a presence or absence of an arrhythmia based on the first electrocardiogram waveform, and
in response to identifying the presence of the arrhythmia, identify the suspected disease based on the first electrocardiogram waveform and the second electrocardiogram waveform.

3. The electronic device of claim 1, wherein the processor is further configured to:
identify a presence or absence of an arrhythmia based on the first electrocardiogram waveform,
identify a presence or absence of a symptom associated with the first electrocardiogram waveform, and
in response to identifying the presence of the arrhythmia and the presence of the symptom, identify the suspected disease based on the first electrocardiogram waveform and the second electrocardiogram waveform.

4. The electronic device of claim 1, wherein the suspected disease includes one of hypercalcemia, hyperkalemia, dehydration, rehydration, hyperglycemia, or
hypoglycemia.

5. The electronic device of claim 1, further comprising a second biometric sensor,
wherein the processor is further configured to obtain the second biometric sensing information using the second biometric sensor.

6. The electronic device of claim 1, further comprising a communication circuit,
wherein the processor is further configured to obtain the second biometric sensing information from an external device through communication with the external device by using the communication circuit.

7. The electronic device of claim 6, wherein the processor is further configured to:
obtain medical information from a medical information server through communication with the medical information server by using the communication circuit, and
obtain the second biometric sensing information from the medical information.

8. The electronic device of claim 1,
wherein the first electrocardiogram waveform and the second electrocardiogram waveform include parameters respectively, the parameters including at least one of a P wave, an RR interval, an R wave, a QRS complex, a PR interval, a PR segment, an ST segment, an ST interval, or a TP interval, and
wherein the processor is further configured to identify a first plurality of parameter sets based on the parameters of the first electrocardiogram waveform and identify a second plurality of parameter sets based on the parameters of the second electrocardiogram waveform.

9. The electronic device of claim 8, wherein the processor is further configured to:
compare each of the first plurality of parameter sets of the first electrocardiogram waveform with each of the second plurality of parameter sets of the second electrocardiogram waveform,
identify a parameter set having a value changed by more than a specified threshold change value from a value of the parameter set of the second electrocardiogram waveform from among the first plurality of parameter sets of the first electrocardiogram waveform, and
identify a suspected disease corresponding to the identified parameter set.

10. The electronic device of claim 8, wherein the first plurality of parameter sets include at least one of:
at least one parameter set among a first parameter set associated with atrial fibrillation,
a second parameter set associated with dehydration,
a third parameter set associated with blood pressure, or
a fourth parameter set associated with blood glucose.

11. A method for providing electrocardiogram-based health information in an electronic device, the method comprising:
obtaining first biometric sensing information comprising a first electrocardiogram waveform using a first biometric sensor comprising a plurality of electrodes and a measurement sensor;
identifying a suspected disease based on the first electrocardiogram waveform and a previously obtained second electrocardiogram waveform;
obtaining second biometric sensing information corresponding to the identified suspected disease; and
displaying, on a display, suspected disease information obtained based on the second biometric sensing information.

12. The method of claim 11, further comprising:
identifying a presence or absence of an arrhythmia based on the first electrocardiogram waveform; and
identifying the suspected disease based on the first electrocardiogram waveform and the second electrocardiogram waveform in response to identifying the presence of the arrhythmia.

13. The method of claim 11, further comprising:
identifying a presence or absence of an arrhythmia based on the first electrocardiogram waveform;
identifying a presence or absence of a symptom associated with the first electrocardiogram waveform; and
in response to identifying the presence of the arrhythmia and the presence of the symptom, identifying the suspected disease based on the first electrocardiogram waveform and the second electrocardiogram waveform.

14. The method of claim 11, wherein the suspected disease includes one of hypercalcemia, hyperkalemia, dehydration, rehydration, hyperglycemia, or hypoglycemia.

15. The method of claim 11, further comprising obtaining the second biometric sensing information using a second biometric sensor included in the electronic device.

16. The method of claim 11, further comprising obtaining the second biometric sensing information through communication with an external medical information server or an external device by using a communication circuit.

17. The method of claim 11,
wherein the first electrocardiogram waveform and the second electrocardiogram waveform comprise parameters respectively, the parameters comprising at least one of a P wave, an RR interval, an R wave, a QRS complex, a PR interval, a PR segment, an ST segment, an ST interval, or a TP interval, and
wherein the method further comprises:
identifying a first plurality of parameter sets based on the parameters of the first electrocardiogram waveform; and
identify a second plurality of parameter sets based on the parameters of the second electrocardiogram waveform.

18. The method of claim 17, further comprising:
comparing each of the first plurality of parameter sets of the first electrocardiogram waveform with each of the second plurality of parameter sets of the second electrocardiogram waveform;
identifying a parameter set having a value changed by more than a specified threshold change value from a value of the parameter set of the second electrocardiogram waveform from among the first plurality of parameter sets of the first electrocardiogram waveform; and
identifying a suspected disease corresponding to the identified parameter set.

19. The method of claim 18, wherein the first plurality of parameter sets comprise at least one of:
at least one parameter set among a first parameter set associated with atrial fibrillation;
a second parameter set associated with dehydration;
a third parameter set associated with blood pressure; or
a fourth parameter set associated with blood glucose.

20. A non-volatile storage medium storing instructions, wherein the instructions are configured to, when executed by an electronic device, cause the electronic device to perform at least one operation, the at least one operation comprising:
obtaining first biometric sensing information comprising a first electrocardiogram waveform using a first biometric sensor comprising an electrode and a measurement sensor;
identifying a suspected disease based on the first electrocardiogram waveform and a previously obtained second electrocardiogram waveform;
obtaining second biometric sensing information corresponding to the identified suspected disease; and
displaying, on a display, suspected disease information obtained based on the second biometric sensing information.
